(19) United States
(12) Patent Application Publication
(10) Pub. No.: US 2004/0241694 A1
(43) Pub. Date: Dec. 2, 2004

(54) Diagnostic and therapeutic use of f-box proteins for alzheimer's disease and related neurodegenerative disorders

(76) Inventors: Heinz Von der Kammer, Hamburg (DE); Johannes Pohlner, Hamburg (DE); Rainer Hipfel, Hamburg (DE)

Correspondence Address: JACOBSON HOLMAN PLLC, 400 SEVENTH STREET N.W., SUITE 600, WASHINGTON, DC, 20004, US

(21) Appl. No.: 10/488,776

(22) PCT Filed: Sep. 7, 2002
(86) PCT No.: PCT/EP02/10057

(30) Foreign Application Priority Data
Sep. 7, 2001 (EP) 01121442.6

(51) Int. Cl. C12Q 1/68; G01N 33/53; G01N 33/567

(52) U.S. Cl. 435/6; 435/7.2

(57) ABSTRACT

The present invention discloses the differential expression of the gene coding for F-box and leucine-rich repeat protein FBL2 in specific brain regions of Alzheimer's disease patients. Based on this finding, the invention provides a method for diagnosing or prognosticating Alzheimer's disease in a subject, or for determining whether a subject is at increased risk of developing Alzheimer's disease. Furthermore, the invention provides therapeutic and prophylactic methods for treating or preventing Alzheimer's disease and related neurodegenerative disorders using a gene coding for an F-box and leucine-rich repeat protein, in particular FBL2. A method of screening for modulating agents of neurodegenerative diseases is also disclosed.

The present invention relates to methods of diagnosing, prognosticating and monitoring the progression of neurodegenerative diseases in a subject. Furthermore, methods of therapy control and screening for modulating agents of neurodegenerative diseases are provided. The invention also discloses pharmaceutical compositions, kits and recombinant animal models.

Neurodegenerative diseases, in particular Alzheimer's disease (AD), have a severely debilitating impact on a patient's life. Furthermore, these diseases constitute an enormous health, social, and economic burden. AD is the most common age-related neurodegenerative condition affecting about 10% of the population over 65 years of age and up to 45% over age 85 (for a recent review see Vickers et al., Progress in Neurobiology 2000, 60:139-165). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brains of individuals suffering from AD are senile plaques, composed of amyloid-β protein, and profound cytoskeletal changes coinciding with the appearance of abnormal filamentous structures and the formation of neurofibrillary tangles. AD is a progressive disease that is associated with early deficits in memory formation and ultimately leads to the complete erosion of higher cognitive function. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10:184-192).

Currently, there is no cure for AD, nor is there an effective treatment to halt the progression of AD or even a method to diagnose AD ante-mortem with high probability. Several risk factors have been identified that predispose an individual to develop AD, among them most prominently the epsilon4 allele of apolipoprotein E (ApoE). Although there are rare examples of early-onset AD which have been attributed to genetic defects in the genes for APP, presenilin-1, and presenilin-2, the prevalent form of late-onset sporadic AD is of hitherto unknown etiologic origin. The late onset and complex pathogenesis of neurodegenerative disorders pose a formidable challenge to the development of therapeutic and diagnostic agents. It is crucial to expand the pool of potential drug targets and diagnostic markers. It is therefore an object of the present invention to provide insight into the pathogenesis of neurodegenerative diseases and to provide methods, materials, and animal models which are suited inter alia for the diagnosis, identification of compounds useful for therapeutic intervention, and development and monitoring of a treatment of these diseases. This object has been solved by the features of the independent claims of the present invention. The subclaims define preferred embodiments.

F-box proteins constitute a family of proteins characterized by an approximately 40 amino-acid motif called the F-box. The F-box motif was first recognized in the cyclin F protein (Bai et al., Cell 1996, 86:263-274). In subsequent studies, at least 38 human F-box proteins have been identified (Kipreos and Pagano. Genome Biology 2000, 1:3002.1-3002.7; Cenciarelli, et al., Current Biology 1999, 9:1177-1179; Winston et al., Current Biology 1999, 9:1180-1182). Frequently, F-box proteins may contain further secondary motifs such as leucine zippers, zinc and ring fingers, cyclin domains, and proline-reach regions. Functionally, the F-box is a peptide module acting as a site of protein-protein interactions. In specific examples, the F-box domain allows proteins to enter into a complex with Skpl (S-phase kinase-associated protein 1). F-box proteins have been implicated in the control of degradation of cellular regulatory proteins via the ubiquitin-proteasome pathway. Protein degradation by the ubiquitin-proteasome pathway is an essential cellular process of selective removal of proteins from the cell and plays a major role in many cellular processes including signal transduction, transcription and the control of the cell cycle (Hershko et al., Nature Medicine 2000, 6:1073-1081; Joazeiro and Hunter, Science 2000, 289:2061; Pickart, Nature Cell Biology 2000, 2:139-141). A key feature of the ubiquitin-proteasome pathway is the covalent attachment of ubiquitin (ubiquitination) by an enzyme complex to a protein destined for removal. Thereby, the ubiquitin-tagged protein is targeted to the 26S proteasome where it becomes proteolytically degraded. In the central nervous system, proteasome-mediated protein degradation is critical for the breakdown and removal of damaged or misfolded cellular proteins (Alves-Rodrigues et al., Trends Neurosci 1998, 21:516-520). The linkage of ubiquitin to a substrate is carried out by the ubiquitin ligase complex, generally comprising three classes of enzymes in a sequential reaction. Ubiquitin activating enzymes (E1) activate ubiquitin by forming a thioester bond between the E1 enzyme and ubiquitin. Subsequently, a transesterification to a member of the E2 class of enzymes (ubiquitin conjugating enzyme) is carried out. Finally, ubiquitin is transferred from the E2 enzyme to the substrate protein with the assistance of a (E3) ubiquitin ligase. E3 ubiquitin ligases are made up of several components. Specifically, F-box proteins are one of the four components of ubiquitin protein ligases (Deshaies, Annu Rev Cell Dev Biol 1999, 15:435-467). The function of F-box proteins within the ubiquitin protein ligase complex may be to specifically recruit substrates for ubiquitin conjugation thus conferring substrate specificity to the degradation process. A possible involvement of the human homolog of the C. elegans F-box protein Sel-10 in Alzheimer's disease has been disclosed recently (WO 0075328). Sel-10 belongs to a subfamily of F-box proteins which is characterized by multiple WD-40 repeats. This subfamily of F-box proteins is referred to as “Fbw” (for a detailed discussion of the domain structure of F-box proteins, giving rise to altogether three subfamilies of F-box proteins, refer to Winston et al., Current Biology 1999, 9:1180-1182).

The subfamily referred to as “Fbx” lacks known protein-interaction domains.

A clearly distinguishable subfamily of F-box proteins contains a C-terminal leucine-rich repeat region in addition to the N-terminally situated F-box motif.

Members of this subfamily of the F-box proteins are referred to as F-box and leucine-rich repeat proteins, “Fbl”. The gene coding for the human F-box and leucine-repeat protein FBL2 was independently identified by several groups (patent applications by Harper and Elledge, WO9918989; Chaiur et al., WO0012679; Zhang et al., WO0075184). Examplary nucleotide sequences of the human FBL2 gene have been deposited in the NCBI Genbank database (accession numbers: NM_{—}012157 (curated version), AF176518 and AF174589 (previous versions)). An examplary amino acid sequence of the corresponding FBL2 protein is referenced in the NCBI Genbank database under the accession number NP_{—}036289 (curated version). Further entries into the database for the FBL2 protein can be found under the NCBI Genbank accession numbers AAF04510 and AAF03128. The rat homolog of the human FBL2 gene exhibits a high degree of similarity to its human counterpart on the amino-acid sequence level (Ilyin et al., FEBS Lett 1999, 459:75-79). The rat FBL2 gene is expressed ubiquitously but with varying amounts among different adult rat tissues. Three different sizes of rat mRNA transcripts were revealed by Northern blot analysis. Whereas the low-sized transcript was predominantly expressed in spleen, thymus and testis, the high molecular weight transcripts were preferably expressed in skeletal muscle, heart and brain. A GFP fusion protein of rat FBL2 expressed in human osteosarcoma U-2OS cells could be localized to punctuate foci in the cytoplasm mainly in the vicinity of the nucleus.

To date, no experiments have been disclosed that demonstrate a relationship between the dysregulation of expression of a gene coding for an F-box and leucine-rich repeat protein, in particular FBL2, and the pathology of neurodegenerative diseases, in particular AD. Such a link, as disclosed in the present invention, offers new ways, inter alia, for the diagnosis and treatment of these diseases.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity. The terms “level” and/or “activity” as used herein further refer to gene expression levels or gene activity. Gene expression can be defined as the utilization of the information contained in a gene by transcription and translation leading to the production of a gene product. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “regulatory elements” shall comprise inducible and non-inducible promoters, enhancers, operators, and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For instance, a “derivative” may be generated by processes such as altered phosphorylation, or glycosylation, or lipidation, or by altered signal peptide cleavage or other types of maturation cleavage. These processes may occur post-translationally. Fragments, derivatives, or variants of the F-box and leucine-rich repeat protein FBL2 may include, but are not limited to, a functional F-box domain or other functional modules, such as leucine-repeat modules, contained within the polypeptide sequence of FBL2. The term “modulator” as used in the present invention and in the claims refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene. The terms “agent”, “reagent”, or “compound” refer to any substance, chemical, composition, or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. The term “variant” as used herein refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention. Furthermore, the term “variant” shall include any shorter or longer version of a polypeptide or protein. “Variants” shall also comprise a sequence that has at least about 80% sequence identity, more preferably at least about 90% sequence identity, and most preferably at least about 95% sequence identity with the amino acid sequences of an F-box and leucine-rich repeat protein, in particular FBL2. “Variants” include, for example, proteins with conservative amino acid substitutions in highly conservative regions. “Proteins and polypeptides” of the present invention include variants, fragments and chemical derivatives of the protein comprising the amino acid sequences of FBL2. They can include proteins and polypeptides which can be isolated from nature or be produced by recombinant and/or synthetic means. Native proteins or polypeptides refer to naturally-occurring truncated or secreted forms, naturally occurring variant forms (e.g. splice-variants) and naturally occurring allelic variants. In the present invention, the terms “risk”, “susceptibility”, and “predisposition” are tantamount and are used with respect to the probability of developing a neurodegenerative disease, preferably Alzheimer's disease. The term ‘AD’ shall mean Alzheimer's disease.

Neurodegenerative diseases or disorders according to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Further conditions involving neurodegenerative processes are, for instance, ischemic stroke, age-related macular degeneration, narcolepsy, motor neuron diseases, prion diseases, traumatic nerve injury and repair, and multiple sclerosis.

In one aspect, the invention features a method of diagnosing or prognosticating a neurodegenerative disease in a subject, or determining whether a subject is at increased risk of developing said disease. The method comprises: determining a level, or an activity, or both said level and said activity of (i) a transcription product of a gene coding for an F-box and leucine-rich repeat protein, and/or of (ii) a translation product of a gene coding for an F-box and leucine-rich repeat protein, and/or of (iii) a fragment, or derivative, or variant of said transcription or translation product in a sample from said subject and comparing said level, and/or said activity to a reference value representing a known disease or health status, thereby diagnosing or prognosticating said neurodegenerative disease in said subject, or determining whether said subject is at increased risk of developing said neurodegenerative disease.

The invention also relates to the construction and the use of primers and probes which are unique to the nucleic acid sequences, or fragments, or derivatives, or variants thereof, as disclosed in the present invention.

Oligonucleotide primers and/or probes can be labeled specifically with fluorescent, bioluminescent, magnetic, or radioactive substances. The invention further relates to the detection and the production of said nucleic acid sequences, or fragments, or derivatives, or variants thereof, using said specific oligonucleotide primers in appropriate combinations. PCR-analysis, a method well known to those skilled in the art, can be performed with said primer combinations to amplify said gene specific nucleic acid sequences from a sample containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimers disease. This feature has utility for developing rapid DNA-based diagnostic tests, preferably also in the format of a kit.

In a further aspect, the invention features a method of monitoring the progression of a neurodegenerative disease in a subject. A level, or an activity, or both said level and said activity, of (i) a transcription product of a gene coding for an F-box and leucine-rich repeat protein, and/or of (ii) a translation product of a gene coding for an F-box and leucine-rich repeat protein, and/or of (iii) a fragment, or derivative, or variant of said transcription or translation product in a sample from said subject is determined. Said level and/or said activity is compared to a reference value representing a known disease or health status. Thereby, the progression of said neurodegenerative disease in said subject is monitored.

In still a further aspect, the invention features a method of evaluating a treatment for a neurodegenerative disease, comprising determining a level, or an activity, or both said level and said activity of (i) a transcription product of a gene coding for an F-box and leucine-rich repeat protein, and/or of (ii) a translation product of a gene coding for an F-box and leucine-rich repeat protein, and/or of (iii) a fragment, or derivative, or variant of said transcription or translation product in a sample obtained from a subject being treated for said disease. Said level, or said activity, or both said level and said activity are compared to a reference value representing a known disease or health status, thereby evaluating the treatment for said neurodegenerative disease.

In a preferred embodiment of the herein and hereinafter claimed methods, kits, recombinant animals, molecules, assays, and uses of the instant invention, said neurodegenerative disease or disorder is Alzheimer's disease, and said subjects suffer from Alzheimer's disease.

In the present invention, it is particularly preferred that said gene coding for an F-box and leucine-rich repeat protein is the gene coding for FBL2 (comprising nucleotide sequences referenced under NCBI Genbank accession numbers: NM_{—}012157, AF176518, AF174589), and that said F-box and leucine-rich repeat protein is FBL2 (comprising amino acid sequences referenced under NCBI Genbank accession number: NP_{—}036289). It should be understood that, within the scope of the instant invention, the referenced nucleotide and amino acid sequences are of examplary nature, and that a gene coding for FBL2, and the corresponding translation products thereof, also comprise fragments, derivatives, and variants of said nucleotide and amino acid sequences. Further currently known members of the subfamily of human genes coding for F-box and leucine-rich repeat proteins and/or members of the subfamily of human F-box and leucine-rich repeat proteins, and/or fragments thereof, are listed in the following with examplary referencing of their corresponding NCBI Genbank/EMBL accession numbers: FBL1 (Skp2), U33761; FLR1, AF142481; FBL3, AF186273, AK001271; FBL3a, AF129532, FBL3b, AF129533; FBL4, AF174590, AF176699, AF199420; FBL5, AF174591, AF176700, AF199355, BC030656; FBL6, AF174592, AF199356; FBL7, AF174593; FBL9, AF176701; FBL10, AK027692; FBL11, BC001203; human homolog to mouse FBL8, AK002140; human homolog to mouse FBL12, AK000195, BC001586, AK027004; hypothetical 43.3 kDa protein, AL133602; P45SKP2-like protein, AF157323; cDNA FLJ20146, AK000153; cDNA FLJ10576, AK001438; FLJ00115 protein, AK024505; cDNA FLJ22888, AK026541; novel protein BA18114.4, AL 121928.

The present invention discloses the detection, differential expression and regulation of a gene coding for an F-box and leucine-rich repeat protein, in particular the F-box and leucine-rich repeat protein FBL2, in specific brain regions of AD patients. Consequently, a gene coding for an F-box and leucine-rich repeat protein and its corresponding gene products may have a causative role in the regional selective neuronal degeneration typically observed in AD. Alternatively, a gene coding for an F-box and leucine-rich repeat protein and its corresponding gene products may confer a neuroprotective function to the remaining surviving nerve cells. Based on these disclosures, the present invention has utility for the diagnostic evaluation and prognosis as well as for the identification of a predisposition to a neurodegenerative disease, in particular AD. Furthermore, the present invention provides methods for the diagnostic monitoring of patients undergoing treatment for such a disease.

It is preferred that the sample to be analyzed and determined is selected from the group comprising brain tissue or other body cells. The sample can also comprise cerebrospinal fluid or other body fluids including saliva, urine, serum plasma, or mucus. Preferably, the methods of diagnosis, prognosis, monitoring the progression or evaluating a treatment for a neurodegenerative disease, according to the instant invention, can be pacticed ex corpore, and such methods preferably relate to samples, for instance, body fluids or cells, removed, collected, or isolated from a subject or patient.

In further preferred embodiments, said reference value is that of a level, or an activity, or both said level and said activity of (i) a transcription product of a gene coding for an F-box and leucine-rich repeat protein, and/or of (ii) a translation product of a gene coding for an F-box and leucine-rich repeat protein, and/or of (iii) a fragment, or derivative, or variant of said transcription or translation product in a sample from a subject not suffering from said neurodegenerative disease.

In preferred embodiments, an alteration in the level and/or activity of a transcription product of a gene coding for an F-box and leucine-rich repeat protein and/or a translation product of a gene coding for an F-box and leucine-rich repeat protein and/or a fragment, or derivative, or variant thereof, in a sample cell, or tissue, or fluid, from said subject relative to a reference value representing a known health status indicates a diagnosis, or prognosis, or increased risk of becoming diseased with a neurodegenerative disease, particularly Alzheimer's disease.

In preferred embodiments, measurement of a level of transcription products of a gene coding for an F-box and leucine-rich repeat protein is performed in a sample from a subject using a quantitative PCR-analysis with primer combinations to amplify said gene-specific sequences from cDNA obtained by reverse transcription of RNA extracted from a sample of a subject. A Northern blot with probes specific for said gene can also be applied. It might further be preferred to measure transcription products by means of chip-based micro-array technologies. These techniques are known to those of ordinary skill in the art (see Sambrook and Russell, Molecular Cloning: A Laboratory Manual, Cold Spring Harbor Laboratory Press, Cold Spring Harbor, N.Y., 2000).

Furthermore, a level and/or activity of a translation product of a gene coding for an F-box and leucine-rich repeat protein, and/or fragment, or derivative, or variant of said translation product, can be detected using an immunoassay, an activity assay, and/or binding assay. These assays can measure the amount of binding between said translation product and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999). All these detection techniques may also be employed in the format of microarrays, protein-arrays, antibody microarrays, or protein-chip based technologies.

In one embodiment of the present invention, it might be preferred to determine an enzymatic activity of a translation product of a gene coding for an F-box and leucine-rich repeat protein, and/or a fragment, or derivative, or variant of said translation product. Specifically, due to an association of F-box and leucine-rich repeat proteins with the ubiquitin-conjugating system, it might be preferred to determine an enzymatic activity of a translation product of a gene coding for an F-box and leucine-rich repeat protein, and/or a fragment, or derivative, or variant of said translation product in an assay for ubiquitin-conjugating enzyme activity, specifically in an assay for E3 ubiquitin-ligase activity. E3 ubiquitin-ligase assays are well known. For instance, such an activity can be determined in a reconstituted system in yeast or in cell-free systems comprising purified or isolated E1 enzyme, E2 enzyme, a translation product of a gene coding for an F-box and leucine-rich repeat protein, and/or a fragment, or derivative, or variant of said translation product, ubiquitin, and an ATP regenerating system (Feldman et al., Cell, 1997; 91:221, Sears et al., J Biol Chem, 1998, 273:1409; US5976849, WO0175145).

In a preferred embodiment, the level, or the activity, or both said level and said activity of (i) a transcription product of a gene coding for an F-box and leucine-rich repeat protein, and/or of (ii) a translation product of a gene coding for an F-box and leucine-rich repeat protein, and/or of (iii) a fragment, or derivative, or variant of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of said subject.

In another aspect, the invention features a kit for diagnosing or prognosticating neurodegenerative diseases, in particular AD, in a subject, or determining the propensity or predisposition of a subject to develop a neurodegenerative disease, in particular AD, said kit comprising:

(a) at least one reagent which is selected from the group consisting of (i) reagents that selectively detect a transcription product of a gene coding for an F-box and leucine-rich repeat protein (ii) reagents that selectively detect a translation product of a gene coding for an F-box and leucine-rich repeat protein; and

(b) instruction for diagnosing, or prognosticating a neurodegenerative disease, in particular AD, or determining the propensity or predisposition of a subject to develop such a disease by

detecting a level, or an activity, or both said level and said activity, of said transcription product and/or said translation product of a gene coding for an F-box and leucine-rich repeat protein, in a sample from said subject; and

diagnosing or prognosticating a neurodegenerative disease, in particular AD, or determining the propensity or predisposition of said subject to develop such a disease, wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status, indicates a diagnosis or prognosis of a neurodegenerative disease, in particular AD, or an increased propensity or predisposition of developing such a disease. The kit, according to the present invention, may be particularly useful for the identification of individuals that are at risk of developing a neurodegenerative disease, in particular AD. Consequently, the kit, according to the present invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular AD in a subject, as well as monitoring success or failure of therapeutic treatment for such a disease of said subject.

In another aspect, the invention features a method of treating or preventing a neurodegenerative disease, in particular Alzheimer's disease, in a subject comprising the administration to said subject in a therapeutically or prophylactically effective amount of an agent or agents which directly or indirectly affect a level, or an activity, or both said level and said activity, of (i) a gene coding for an F-box and leucine-rich repeat protein, and/or (ii) a transcription product of a gene coding for an F-box and leucine-rich repeat protein, and/or (iii) a translation product of a gene coding for an F-box and leucine-rich repeat protein, and/or (iv) a fragment, or derivative, or variant of (i) to (iii). Said agent may comprise a small molecule, or it may also comprise a peptide, an oligopeptide, or a polypeptide. Said peptide, oligopeptide, or polypeptide may comprise an amino acid sequence of a translation product of a gene coding for an F-box and leucine-rich repeat protein, or a fragment, or derivative, or variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of a gene coding for an F-box and leucine-rich repeat protein, either in sense or in antisense orientation.

In preferred embodiments, the method comprises the application of per se known methods of gene therapy and/or antisense nucleic acid technology to administer said agent or agents. In general, gene therapy comprises several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26:274-278 and Mulligan, Science, 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3:743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5:389-395; Agrawal and Akhtar, Trends Biotechnol 1995, 13:197-199; Crooke, Biotechnology 1992, 10:882-6).

Apart from hybridization strategies, the application of ribozymes, i.e. RNA molecules that act as enzymes, destroying RNA that carries the message of disease has also been described (see e.g. Barinaga, Science 1993, 262:1512-1514). In preferred embodiments, the subject to be treated is a human, and therapeutic antisense nucleic acids or derivatives thereof are directed against a human F-box and leucine-rich repeat protein gene, particularly the FBL2 gene. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol, 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can be another effective approach for nucleic acid therapy (Hannon, Nature 2002, 418:244-251).

In further preferred embodiments, the method of treatment comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection or liposomal mediated transfection.

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector. Said agent, particularly a therapeutic protein, can further be administered to said subject by a process comprising the injection or the systemic administration of a fusion protein, said fusion protein being a fusion of a protein transduction domain with said agent.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson DA, Curr. Opin. Pharmacol. 2002, 2: 34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mammal, a mouse, a rat, a fish, a fly, or a worm; a domestic animal, or a non-human primate. The experimental animal can be an animal model for a neurodegenerative disorder, e.g. a transgenic mouse with an AD-type neuropathology.

In a further aspect, the invention features a modulator of an activity, or a level, or both said activity and said level of at least one substance which is selected from the group consisting of (i) a gene coding for an F-box and leucine-rich repeat protein, and/or (ii) a transcription product of a gene coding for an F-box and leucine-rich repeat protein, and/or (iii) a translation product of a gene coding for an F-box and leucine-rich repeat protein, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) a gene coding for an F-box and leucine-rich repeat protein, and/or (ii) a transcription product of a gene coding for an F-box and leucine-rich repeat protein, and/or (iii) a translation product of a gene coding for an F-box and leucine-rich repeat protein, and/or (iv) a fragment, or derivative, or variant of (i) to (iii) for use in a pharmaceutical composition.

In another aspect, the invention provides for the use of a modulator of an activity, or a level, or both said activity and said level of at least one substance which is selected from the group consisting of (i) a gene coding for an F-box and leucine-rich repeat protein, and/or (ii) a transcription product of a gene coding for an F-box and leucine-rich repeat protein, and/or (iii) a translation product of a gene coding for an F-box and leucine-rich repeat protein, and/or (iv) a fragment, or derivative, or variant of (i) to (iii) for a preparation of a medicament for treating or preventing a neurodegenerative disease, in particular AD.

In one aspect, the present invention also provides a kit comprising one or more containers filled with a therapeutically or prophylactically effective amount of said pharmaceutical composition.

In a further aspect, the invention features a recombinant, non-human animal comprising a non-native gene sequence coding for an F-box and leucine-rich repeat protein, or a fragment, or a derivative, or a variant thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of a neurodegenerative disease, in particular AD. Strategies and techniques for the generation and construction of such an animal are known to those of ordinary skill in the art (see e.g. Capecchi, Science, 1989, 244:1288-1292 and Hogan et al., 1994, Manipulating the Mouse Embryo: A Laboratory Manual, Cold Spring Harbor Laboratory Press, Cold Spring Harbor, N.Y.). It is preferred to make use of such a recombinant non-human animal as an animal model for investigating neurodegenerative diseases, in particular AD.

In preferred embodiments, said recombinant, non-human animal comprises a non-native gene sequence coding for the F-box and leucine-rich repeat protein FBL2, or a fragment, or derivative, or variant thereof.

In another aspect, the invention features an assay for screening for a modulator of neurodegenerative diseases, in particular AD, or related diseases and disorders of one or more substances selected from the group consisting of (i) a gene coding for an F-box and leucine-rich repeat protein, and/or (ii) a transcription product of a gene coding for an F-box and leucine-rich repeat protein, and/or (iii) a translation product of a gene coding for an F-box and leucine-rich repeat protein, and/or (iv) a fragment, or derivative, or variant of (i) to (iii). This screening method comprises (a) contacting a cell with a test compound, and (b) measuring the level, or the activity, or both the level and the activity of one or more substances recited in (i) to (iv), and (c) measuring the level, or the activity, or both the level and the activity of said substances in a control cell not contacted with said test compound, and (d) comparing the levels of the substance in the cells of step (b) and (c), wherein an alteration in the level and/or activity of said substances in the contacted cells indicates that the test compound is a modulator of said diseases and disorders.

In one further aspect, the invention features a screening assay for a modulator of neurodegenerative diseases, in particular AD, or related diseases and disorders of one or more substances selected from the group consisting of (i) a gene coding for an F-box and leucine-rich repeat protein, and/or (ii) a transcription product of a gene coding for an F-box and leucine-rich repeat protein, and/or (iii) a translation product of a gene coding for an F-box and leucine-rich repeat protein, and/or (iv) a fragment, or derivative, or variant of (i) to (iii), comprising (a) administering a test compound to a test animal which is predisposed to developing or has already developed symptoms of a neurodegenerative disease or related diseases or disorders, and (b) measuring the level and/or activity of one or more substances recited in (i) to (iv), and (c) measuring the level and/or activity of said substances in a matched control animal which is equally predisposed to developing or has already developed said symptoms of a neurodegenerative disease, and to which animal no such test compound has been administered, and (d) comparing the level and/or activity of the substance in the animals of step (b) and (c), wherein an alteration in the level and/or activity of substances in the test animal indicates that the test compound is a modulator of said diseases and disorders.

In a preferred embodiment, said test animal and/or said control animal is a recombinant, non-human animal which expresses an F-box and leucine-rich repeat protein, in particular FBL2, or a fragment, or a derivative, or a variant thereof, under the control of a transcriptional regulatory element which is not the native gene transcriptional control regulatory element.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. Said modulator may also be identifiable by other assays of screening.

In another aspect, the present invention provides for an assay for testing a compound, preferably for screening a plurality of compounds, for inhibition of binding between a ligand and an F-box and leucine-rich repeat protein, or a fragment, or derivative, or variant thereof. Said screening assay comprises the steps of (i) adding a liquid suspension of said F-box and leucine-rich repeat protein, or a fragment, or derivative, or variant thereof, to a plurality of containers, and (ii) adding a compound, preferably a plurality of compounds, to be screened for said inhibition to said plurality of containers, and (iii) adding detectable ligand, preferably fluorescently detectable ligand, to said containers, and (iv) incubating the liquid suspension of said F-box and leucine-rich repeat protein, or said fragment, or derivative, or variant thereof, and said compounds, and said detectable ligand, and (v) measuring the amounts of detectable ligand or fluorescence associated with said F-box and leucine-rich repeat protein, or with said fragment, or derivative, or variant thereof, and (vi) determining the degree of inhibition by one or more of said compounds of binding of said ligand to said F-box and leucine-rich repeat protein, or said fragment, or derivative, or variant thereof. Instead of utilizing a fluorescently detectable label, it might in some aspects be preferred to use any other detectable label known to the person skilled in the art, e.g. radioactive label, and detect it accordingly. Said method may be useful for the identification of novel compounds as well as for evaluating compounds which have been improved or otherwise optimized in their ability to inhibit the binding of a ligand to an F-box and leucine-rich repeat protein, or a fragment, or derivative, or variant thereof. In one further embodiment, the present invention provides a method for producing a medicament comprising the steps of (i) identifying a compound as an inhibitor of binding between a ligand and a gene product of the gene coding for an F-box and leucine-rich repeat protein by the aforementioned method of inhibitory binding assay and (ii) admixing the compound with a pharmaceutical carrier. Said compound may also be identifiable by other types of screening assays.

In another aspect, the invention features an assay for testing a compound, preferably for screening a plurality of compounds, to determine the degree of binding of said compound or compounds to an F-box and leucine-rich repeat protein, or to a fragment, or derivative, or variant thereof. Said screening assay comprises the steps of (i) adding a liquid suspension of said F-box and leucine-rich repeat protein, or a fragment, or derivative, or variant thereof, to a plurality of containers, and (ii) adding a detectable compound, preferably a plurality of detectable compounds, in particular fluorescently detectable compounds, to be screened for said binding to said plurality of containers, and (iii) incubating the liquid suspension of said F-box and leucine-rich repeat protein, or said fragment, or derivative, or variant thereof, and said detectable compound, preferably said plurality of detectable compounds, and (iv) measuring the amounts of detectable compound or fluorescence associated with said F-box and leucine-rich repeat protein, or with said fragment, or derivative, or variant thereof, and (v) determining the degree of binding by one or more of said compounds to said F-box and leucine-rich repeat protein, or said fragment, or derivative, or variant thereof. In this type of assay it might be preferred to use a fluorescent label. However, any other type of detectable label might also be employed. Said method may be useful for the identification of novel compounds as well as for evaluating compounds which have been improved or otherwise optimized in their ability to bind to an F-box and leucine-rich repeat protein.

In one further embodiment, the present invention provides a method for producing a medicament comprising the steps of (i) identifying a compound as a binder to an F-box and leucine-rich repeat protein by the aforementioned binding assays and (ii) admixing the compound with a pharmaceutical carrier.

Said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

In all types of assays disclosed herein it is preferred to study and conduct screening assays with the F-box and leucine-rich repeat protein FBL2.

The present invention features a protein molecule, said protein molecule being a translation product of a gene coding for an F-box and leucine-rich repeat protein, or a fragment, or derivative, or variant thereof, for use as a diagnostic target for detecting a neurodegenerative disease, in particular Alzheimer's disease.

The present invention further features a protein molecule, said protein molecule being a translation product of the gene coding for an F-box and leucine-rich repeat protein, or a fragment, or derivative, or variant thereof, for use as a screening target for reagents or compounds preventing, or treating, or ameliorating a neurodegenerative disease, in particular Alzheimer's disease.

The present invention features an antibody which is specifically immunoreactive with an immunogen, wherein said immunogen is a translation product of a gene coding for an F-box and leucine-rich repeat protein, or a fragment, or derivative, or variant thereof. The immunogen may comprise immunogenic or antigenic epitopes or portions of a translation product of said gene, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y.). The term “antibody”, as employed in the present invention, encompasses all forms of antibodies known in the art, such as polyclonal, monoclonal, chimeric, recombinatorial, anti-idiotypic, humanized, or single chain antibodies, as well as fragments thereof. Antibodies of the present invention are useful, for instance, in a variety of diagnostic and therapeutic methods involving detecting translation products of a gene coding for an F-box and leucine-rich repeat protein.

In a preferred embodiment of the present invention, said antibodies can be used for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to AD. Immunocytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in U.S. Pat. No. 6,150,173.

Other features and advantages of the invention will be apparent from the following description of figures and examples which are illustrative only and not intended to limit the remainder of the disclosure in any way.

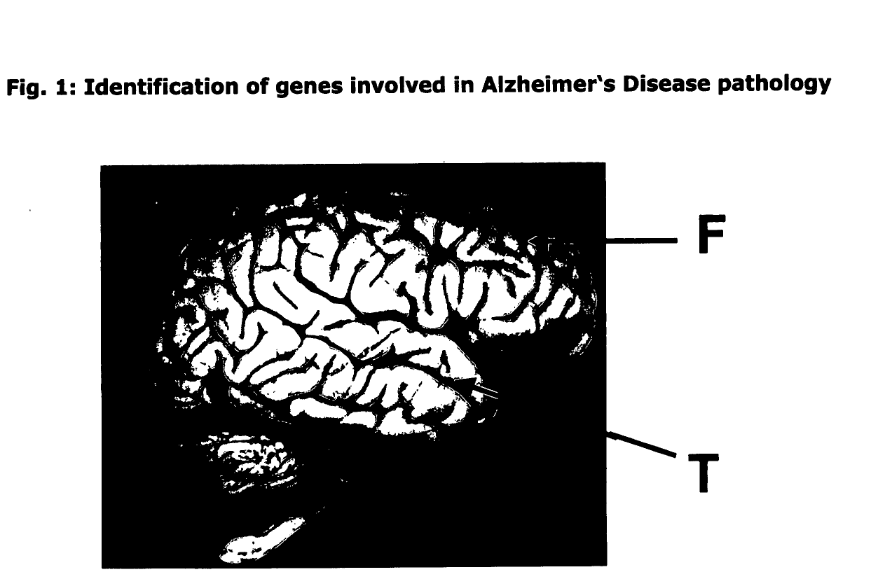

FIG. 1 depicts the brain regions with selective vulnerability to neuronal loss and degeneration in AD. Primarily, neurons within the inferior temporal lobe, the entorhinal cortex, the hippocampus, and the amygdala are subject to degenerative processes in AD (Terry et al., Annals of Neurology 1981, 10:184-192). These brain regions are mostly involved in the processing of learning and memory functions. In contrast, neurons within the frontal cortex, the occipital cortex, and the cerebellum remain largely intact and preserved from neurodegenerative processes in AD. Brain tissues from the frontal cortex (F) and the temporal cortex (T) of AD patients and healthy, age-matched control individuals were used for the herein disclosed examples. For illustrative purposes, the image of a normal healthy brain was taken from a publication by Strange (Brain Biochemistry and Brain Disorders, Oxford University Press, Oxford, 1992, p.4).

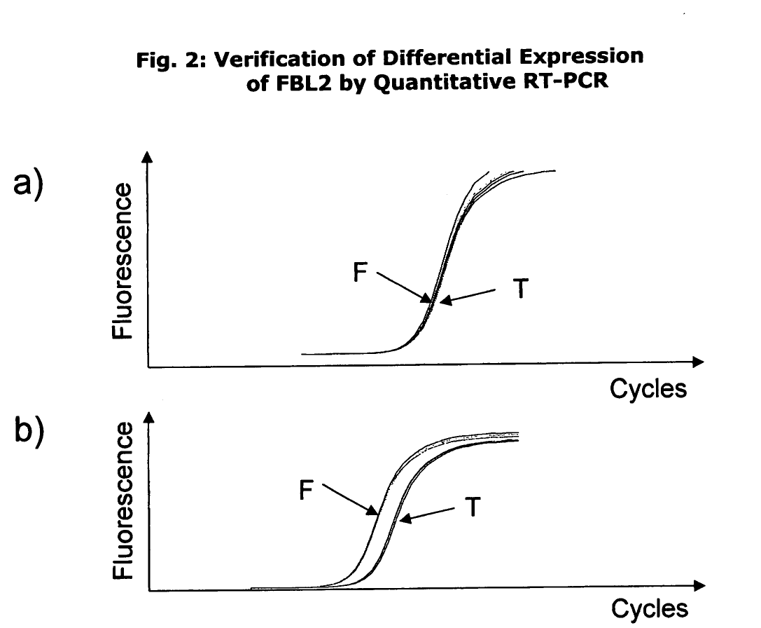

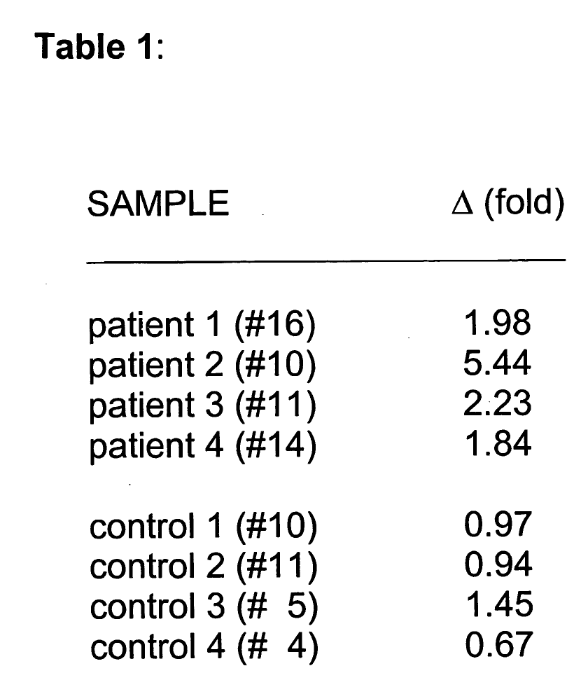

FIG. 2 illustrates the verification of the differential expression of the FBL2 gene by quantitative RT-PCR analysis. Quantification of RT-PCR products from RNA samples collected from the frontal cortex (F) and temporal cortex (T) of healthy, age-matched control individuals (FIG. 2a) and AD patients (FIG. 2b) was performed by the LightCycler rapid thermal cycling technique. The data were normalized to the combined average values of a set of standard genes which showed no significant differences in their gene expression levels. Said set of standard genes consisted of genes for the ribosomal protein S9, the transferrin receptor, GAPDH, and beta-actin. The figure depicts the kinetics of amplification by plotting the cycle number against the amount of amplified material as measured by its fluorescence. The amplification kinetics of FBL2 cDNA from both the frontal and temporal cortices of a normal control individual during the exponential phase of the reaction overlap, whereas in Alzheimer's disease there is a significant separation of the curves for the samples derived from frontal and temporal cortex, which is indicative of an up-regulation of FBL2 gene expression in frontal cortex relative to temporal cortex.

Table 1 lists the gene expression levels in the frontal cortex relative to the temporal cortex for the FBL2 gene in four AD patients (1.84 to 5.44 fold) and four healthy, age-matched control individuals (0.67 to 1.45 fold). The values shown are reciprocal values according to the formula described in present invention.

### EXAMPLE I

Brain Tissue Dissection From Patients With Alzheimer's Disease:

Brain tissues from AD patients and age-matched control subjects were collected on average within 5 hours post-mortem and immediately frozen on dry ice. Sample sections from each tissue were fixed in paraformaldehyde for subsequent histopathological confirmation of the diagnosis. Brain areas for differential expression analysis were identified (see FIG. 1) and stored at minus 80° C. until RNA extractions were performed.

(i) Isolation of Total mRNA:

Total RNA was extracted from post-mortem brain tissue by using the RNeasy kit (Qiagen) according to the manufacturer's protocol. The quality of the prepared RNA was determined by formaldehyde agarose gel electrophoresis and Northern blotting according to standard procedures (Sambrook and Russell, Molecular Cloning: A Laboratory Manual, Cold Spring Harbor Laboratory Press, Cold Spring Harbor, N.Y., 2000). The accurate RNA concentration and RNA quality were also determined with the DNA LabChip system using the Agilent 2100 Bioanalyzer (Agilent Technologies). For additional quality testing of the prepared RNA, i.e. testing for partial degradation and genomic DNA contamination, specifically designed intronic GAPDH oligonucleotides and genomic DNA as reference control were utilized to generate a melting curve with the LightCycler technology as described in the protocol supplied by the manufacturer (Roche).

(iii) cDNA synthesis and identification of differentially expressed genes by suppressive subtractive hybridization: This technique compares two populations of mRNA and provides clones of genes that are expressed in one population but not in the other. The applied technique was described in detail by Diatchenko et al. (Proc Natl Acad Sci USA 1996, 93:6025-30). In the present invention, mRNA populations derived from different brain regions of AD patients were compared. Specifically, mRNA of the frontal cortex was subtracted from mRNA of the inferior temporal cortex. The necessary reagents were taken from the PCR-Select cDNA subtraction kit (Clontech), and all steps were performed as described in the manufacturer's protocol. Specifically, 2 μg mRNA each were used for first-strand and second-strand cDNA synthesis. After Rsal-digestion and adaptor ligation, hybridization of tester and driver was performed for 8 hours (first hybridization) and 15 hours (second hybridization) at 68° C. Two PCR steps were performed to amplify differentially expressed genes (first PCR: 27 cycles of 94° C. and 30 sec, 66° C. and 30 sec, and 72° C. and 1.5 min; nested PCR: 12 cycles of 94° C. and 30 sec, 66° C. and 30 sec, and 72° C. and 1.5 min) using adaptor specific primers (included in the subtraction kit) and 50×Advantage Polymerase Mix (Clontech). Efficiencies of Rsal-digestions, adaptor ligations and subtractive hybridizations were checked as recommended in the kit. Subtracted cDNAs were inserted into the pCR-vector and transformed into E. coli INVαF′ cells (Invitrogen). To isolate individual cDNAs of the subtracted library, single bacterial transformants were incubated in 100 μl LB (with 50 μg/ml ampicillin) at 37° C. for at least 4 hours. Inserts were PCR amplified (95° C. and 30 sec, 68° C. and 3 min for 30 cycles) in a volume of 20 pi containing 10 mM Tris-HCl pH 9.0, 1.5 mM MgCl₂, 50 mM KCl, 200 μM dNTP, 0.5 μM adaptor specific primers (included in the subtraction kit), 1.5 Units Taq polymerase (Pharmacia Biotech), and 1 μl of bacterial culture. An aliquot of the mixture (1.5 μl) containing 3 μl PCR amplified inserts and 2 μl 0.3 N NaOH/15% Ficoll were spotted onto a positively charged nylon membrane (Roche). In this way, hundreds of spots were arrayed on duplicate filters for subsequent hybridization. The differential screening step consisted of hybridizations of the subtracted library with itself to minimize background (Wang and Brown, Proc Natl Acad Sci USA 1991, 88:11505-9). The probes were made of the nested PCR product of the subtraction following the instructions of the Clontech subtraction kit. Labeling with digoxigenin was performed with the DIG DNA Labeling Kit (Roche). Hybridizations were carried out overnight in DIG Easy HYB (Roche) at 43° C. The filters were washed twice in 2×SSC/0.5% SDS at 68° C. for 15 min and twice in 0.1×SSC/0.5% SDS at 68° C. for 15 min, and subjected to detection using anti-DIG-AP conjugates and CDP-Star as chemiluminescent substrate according to the instructions of the DIG DNA Detection Kit (Roche). Blots were exposed to Kodak Biomax MR chemiluminescent film at room temperature for several minutes. The nucleotide sequences of clones of interest were obtained using methods well known to those skilled in the art. For nucleotide sequence analysis, alignments, and homology searches, computer algorithms of the University of Wisconsin Genetics Computer Group (GCG) in conjunction with publicly available nucleotide and protein sequence information (NCBI Genbank and EMBL databases) were employed.

(ii) Confirmation of Differential Expression by Quantitative RT-PCR:

Positive corroboration of differential expression of the FBL2 gene was performed using the LightCycler technology (Roche). This technique features rapid thermal cyling for the polymerase chain reaction as well as real-time measurement of fluorescent signals during amplification and therefore allows for highly accurate quantification of RT-PCR products by using a kinetic rather than an endpoint approach. The ratio of FBL2 cDNA from the frontal cortex versus temporal cortex was determined (relative quantification). First, a standard curve was generated to determine the efficiency of the PCR with specific primers for FBL2 (5′-TACTGGGTGGAGCAGGGTCTT-3′ and 5′-GGTCCCTGGAGGTGTATATGACA-3′). PCR amplification (95° C. and 1 sec, 56° C. and 5 sec, and 72° C. and 5 sec) was performed in a volume of 20 μl containing Lightcycler-DNA-Master-SYBR-Green mix (containing Taq DNA polymerase, reaction buffer, dNTP mix with dUTP instead of dTTP, SYBR Green I dye, and 1 mM MgCl₂, Roche), additionally containing 3 mM MgCl₂, 0.5 μM primers, 0.16 μl TaqStart antibody (Clontech), and 1 μl of a cDNA dilution series (40, 20, 10, 5, and 1 ng human total brain cDNA, Clontech).

Melting curve analysis revealed a single peak at approximately 83° C. with no visible primer dimers. Quality and size of the PCR product were determined by agarose gel electrophoresis and DNA LabChip analysis (Agilent 2100 Bioanalyzer, Agilent Technologies). A single band of the expected size 66 bp was observed.

In an analogous manner, the above described PCR protocol (with indicated exceptions) was applied to determine the PCR efficiency of a set of reference genes which were selected as a reference standard for quantification. In the present invention, the mean value of five such reference genes was determined: (1) cyclophilin B, using the specific primers 5′-ACTGAAGCACTACGGGCCTG-3′ and 5′-AGCCGTTGGTGTCTTTGCC-3′, (exception: an additional 1 mM MgCl₂ was added instead of 3 mM). Melting curve analysis revealed a single peak at approximately 87° C. with no visible primer dimers. Agarose gel analysis of the PCR product showed one single band of the expected size (62 bp). (2) Ribosomal protein S9 (RPS9), using the specific primers 5′-GGTCAAATTTACCCTGGCCA-3′ and 5′-TCTCATCAAGCGTCAGCAGTTC-3′ (exception: an additional 1 mM MgCl₂ was added instead of 3 mM). Melting curve analysis revealed a single peak at approximately 85° C. with no visible primer dimers. Agarose gel analysis of the PCR product showed one single band with the expected size (62 bp). (3) beta-actin, using the specific primers 5′-TGGAACGGTGAAGGTGACA-3′ and 5′-GGCAAGGGACTTCCTGTAA-3′. Melting curve analysis revealed a single peak at approximately 87° C. with no visible primer dimers. Agarose gel analysis of the PCR product showed one single band with the expected size (142 bp). (4) GAPDH, using the specific primers 5′-CGTCATGGGTGTGAACCATG-3′ and 5′-GCTAAGCAGTTGGTGGTGCAG-3′. Melting curve analysis revealed a single peak at approximately 83° C. with no visible primer dimers. Agarose gel analysis of the PCR product showed one single band with the expected size (81 bp). (5) Transferrin receptor TRR, using the specific primers 5′-GTCGCTGGTCAGTTCGTGATT-3′ and 5′-AGCAGTTGGCTGTTGTACCTCTC-3′. Melting curve analysis revealed a single peak at approximately 83° C. with no visible primer dimers. Agarose gel analysis of the PCR product showed one single band with the expected size (80 bp).

For calculation of the values, first the logarithm of the cDNA concentration was plotted against the threshold cycle number Ct for FBL2 and the five reference standard genes. The slopes and the intercepts of the standard curves (i.e. linear regressions) were calculated for all genes. In a second step, cDNAs from temporal cortex and frontal cortex were analyzed in parallel and normalized to cyclophilin B. The Cₜ values were measured and converted to ng total brain cDNA using the corresponding standard curves:

10\hat{ }((Cₜ value−intercept)/slope) [ng total brain cDNA]

The values of temporal and frontal cortex FBL2 cDNAs were normalized to cyclophilin B, and the ratio was calculated using the following formula:

$$Ratio = \frac{FBL2 temporal [ ng ] / cyclophilin B temporal [ ng ]}{FBL2 frontal [ ng ] / cyclophilin B frontal [ ng ]}$$

In a third step, the set of reference standard genes was analyzed in parallel to determine the mean average value of the temporal to frontal ratios of expression levels of the reference standard genes for each individual brain sample. As cyclophilin B was analyzed in step 2 and step 3, and the ratio from one gene to another gene remained constant in different runs, it was possible to normalize FBL2 to the mean average value of the set of reference standard genes instead of normalizing to one single gene alone. The calculation was performed by dividing the ratio shown above by the deviation of cyclophilin B from the mean value of all housekeeping genes. The results of one such quantitative RT-PCR analysis for the FBL2 gene are shown in FIG. 2.

1. A method of diagnosing or prognosticating a neurodegenerative disease in a subject, or determining whether a subject is at increased risk of developing said disease, comprising determining a level and/or an activity of
(i) a transcription product of a gene coding for the F-box protein FBL2 and, and/or
(ii) a translation product of a gene coding for the F-box protein FBL2, and/or
(iii) a fragment, or derivative, or variant of said transcription or translation product,
in a sample from said subject and comparing said level and/or said activity to a reference value representing a known disease or health status, thereby diagnosing or prognosticating said neurodegenerative disease in said subject, or determining whether said subject is at increased risk of developing said neurodegenerative disease.

2. A method of monitoring the progression of a neurodegenerative disease in a subject, comprising determining a level and/or an activity of
(i) a transcription product of a gene coding for the F-box protein FBL2, and/or
(ii) a translation product of a gene coding for the F-box protein FBL2, and/or
(iii) a fragment, or derivative, or variant of said transcription or translation product,
in a sample from said subject and comparing said level and/or said activity to a reference value representing a known disease or health status, thereby monitoring the progression of said neurodegenerative disease in said subject.

3. A method of evaluating a treatment for a neurodegenerative disease, comprising determining a level and/or an activity of
(i) a transcription product of a gene coding for the F-box protein FBL2, and/or
(ii) a translation product of a gene coding for the F-box protein FBL2, and/or
(iii) a fragment, or derivative, or variant of said transcription or translation product,
in a sample from a subject being treated for said disease and comparing said level and/or said activity to a reference value representing a known disease or health status, thereby evaluating said treatment for said neurodegenerative disease.

4. The method according to claim 1 wherein said neurodegenerative disease is Alzheimer's disease.

5. The method according to claim 1 wherein said sample comprises a cell, or a tissue, or a body fluid, in particular cerebrospinal fluid.

6. The method according to claim 1 wherein said reference value is that of a level and/or an activity of
(i) a transcription product of a gene coding for the F-box protein FBL2, and/or
(ii) a translation product of a gene coding for the F-box protein FBL2, and/or
(iii) a fragment, or derivative, or variant of said transcription or translation product,
in a sample from a subject not suffering from said neurodegenerative disease.

7. The method according to claim 1 wherein an alteration in the level and/or activity of a transcription product of a gene coding for the F-box protein FBL2 and/or a translation product of a gene coding for the F-box protein FBL2 and/or a fragment, or derivative, or variant thereof, in a sample cell, or tissue, or body fluid, in particular cerebrospinal fluid, from said subject relative to a reference value representing a known health status indicates a diagnosis, or prognosis, or increased risk of Alzheimer's disease in said subject.

8. The method according to claim 1, further comprising comparing a level and/or an activity of
(i) a transcription product of a gene coding for the F-box protein FBL2, and/or
(ii) a translation product of a gene coding for the F-box protein FBL2, and/or
(iii) a fragment, or derivative, or variant of said transcription or translation product, in a series of samples taken from said subject over a period of time.

9. The method according to claim 8 wherein said subject receives a treatment prior to one or more of said sample gatherings.

10. The method according to claim 9 wherein said level and/or activity is determined before and after said treatment of said subject.

11. A kit for diagnosing or prognosticating a neurodegenerative disease, in particular Alzheimer's disease, in a subject, or determining the propensity or predisposition of a subject to develop such a disease, said kit comprising:
(a) at least one reagent which is selected from the group consisting of
(i) reagents that selectively detect a transcription product of a gene coding for the F-box protein FBL2 and (ii) reagents that selectively detect a translation product of a gene coding for the F-box protein FBL2, and
(b) an instruction for diagnosing or prognosticating a neurodegenerative disease, in particular Alzheimer's disease, or determining the propensity or predisposition of a subject to develop such a disease by (i) detecting a level, or an activity, or both said level and said activity, of said transcription product and/or said translation product of a gene coding for the F-box protein FBL2 in a sample from said subject; and (ii) diagnosing or prognosticating a neurodegenerative disease, in particular Alzheimer's disease, or determining the propensity or predisposition of said subject to develop such a disease, wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status indicates a diagnosis or prognosis of a neurodegenerative disease, in particular Alzheimer's disease, or an increased propensity or predisposition of developing such a disease.

12. A method of treating or preventing a neurodegenerative disease, in particular Alzheimer's disease, in a subject comprising administering to said subject in a therapeutically or prophylactically effective amount an agent or agents which directly or indirectly affect an activity and/or a level of (i) a gene coding for the F-box protein FBL2, and/or (ii) a transcription product of a gene coding for the F-box protein FBL2, and/or (iii) a translation product of a gene coding for the F-box protein FBL2, and/or (iv) a fragment, or derivative, or variant of (i) to (iii).

13. A modulator of an activity and/or of a level of at least one substance which is selected from the group consisting of (i) a gene coding for the F-box protein FBL2, and/or (ii) a transcription product of a gene coding for the F-box protein FBL2, and/or (iii) a translation product of a gene coding for the F-box protein FBL2, and/or (iv) a fragment, or derivative, or variant of (i) to (iii).

14. A pharmaceutical composition comprising a modulator according to claim 13.

15. A modulator of an activity and/or of a level of at least one substance which is selected from the group consisting of (i) a gene coding for the F-box protein FBL2, and/or (ii) a transcription product of a gene coding for the F-box protein FBL2, and/or (iii) a translation product of a gene coding for the F-box protein FBL2, and/or (iv) a fragment, or derivative, or variant of (i) to (iii) for use in a pharmaceutical composition.

16. Use of a modulator of an activity and/or of a level of at least one substance which is selected from the group consisting of (i) a gene coding for the F-box protein FBL2, and/or (ii) a transcription product of a gene coding for the F-box protein FBL2, and/or (iii) a translation product of a gene coding for the F-box protein FBL2, and/or (iv) a fragment, or derivative, or variant of (i) to (iii) for a preparation of a medicament for treating or preventing a neurodegenerative disease, in particular Alzheimer's disease.

17. A kit, comprising in one or more containers, a therapeutically or prophylactically effective amount of the pharmaceutical composition of claim 14.

18. A recombinant, non-human animal comprising a non-native gene sequence coding for the F-box protein FBL2, or a fragment, or a derivative, or a variant thereof, said animal being obtainable by:
(i) providing a gene targeting construct comprising said gene sequence and a selectable marker sequence, and
(ii) introducing said targeting construct into a stem cell of a non-human animal, and
(iii) introducing said non-human animal stem cell into a non-human embryo, and
(iv) transplanting said embryo into a pseudopregnant non-human animal, and
(v) allowing said embryo to develop to term, and
(vi) identifying a genetically altered non-human animal whose genome comprises a modification of said gene sequence in both alleles, and
(vii) breeding the genetically altered non-human animal of step (vi) to obtain a genetically altered non-human animal whose genome comprises a modification of said endogenous gene, wherein said disruption results in said non-human animal exhibiting a predisposition to developing symptoms of a neurodegenerative disease or related disease or disorders.

19. Use of the recombinant, non-human animal according to claim 18 as an animal model for investigating neurodegenerative diseases, in particular Alzheimer's disease.

20. An assay for screening for a modulator of neurodegenerative diseases, in particular Alzheimer's disease, or related diseases or disorders of one or more substances selected from the group consisting of
(i) a gene coding for the F-box protein FBL2, and/or
(ii) a transcription product of a gene coding for the F-box protein FBL2, and/or
(iii) a translation product of a gene coding for the F-box protein FBL2, and/or
(iv) a fragment, or derivative, or variant of (i) to (iii), said method comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and
(d) comparing the levels and/or activities of the substance in the cells of step (b) and (c), wherein an alteration in the activity and/or level of substances in the contacted cells indicates that the test compound is a modulator of said diseases or disorders.

21. A method of screening for a modulator of neurodegenerative diseases, in particular Alzheimer's disease, or related diseases or disorders of one or more substances selected from the group consisting of
(i) a gene coding for the F-box protein FBL2, and/or
(ii) a transcription product of a gene coding for the F-box protein FBL2, and/or
(iii) a translation product of a gene coding for the F-box protein FBL2, and/or
(iv) a fragment, or derivative, or variant of (i) to (iii), said method comprising:
(a) administering a test compound to a test animal which is predisposed to developing or has already developed symptoms of a neurodegenerative disease or related diseases or disorders in respect of the substances recited in (i) to (iv);
(b) measuring the activity and/or level of one or more substances recited in (i) to (iv);
(c) measuring the activity and/or level of one or more substances recited in (i) or (iv) in a matched control animal which is predisposed to developing or has already developed symptoms of a neurodegenerative disease or related diseases or disorders in respect to the substances recited in (i) to (iv) and to which animal no such test compound has been administered;
(d) comparing the activity and/or level of the substance in the animals of step (b) and (c), wherein an alteration in the activity and/or level of substances in the test animal indicates that the test compound is a modulator of said diseases or disorders.

22. The method according to claim 21 wherein said test animal and/or said control animal is a recombinant animal which expresses the F-box protein FBL2, or a fragment, or derivative, or variant thereof, under the control of a transcriptional control element which is not the native gene transcriptional control element.

23. A method of testing a compound, preferably of screening a plurality of compounds, for inhibition of binding between a ligand and the F-box protein FBL2, or a fragment, or derivative, or variant thereof, said method comprising the steps of:
(i) adding a liquid suspension of said F-box protein FBL2, or a fragment, or derivative, or variant thereof, to a plurality of containers;
(ii) adding a compound, preferably a plurality of compounds, to be screened for said inhibition of binding to said plurality of containers;
(iii) adding a detectable ligand, in particular a fluorescently detectable ligand, to said containers;
(iv) incubating the liquid supension of said F-box protein FBL2, or said fragment, or derivative, or variant thereof, and said compound, preferably said plurality of compounds, and said ligand;
(v) measuring amounts of detectable ligand or fluorescence associated with said F-box protein FBL2, or with said fragment, or derivative, or variant thereof; and
(vi) determining the degree of inhibition by one or more of said compounds of binding of said ligand to said F-box protein FBL2, or said fragment, or derivative, or variant thereof.

24. A method of testing a compound, preferably of screening a plurality of compounds, to determine the degree of binding of said compound or compounds to the F-box protein FBL2, or to a fragment, or derivative, or variant thereof, said method comprising the steps of:
(i) adding a liquid suspension of said F-box protein FBL2, or a fragment, or derivative, or variant thereof, to a plurality of containers;
(ii) adding a detectable compound, preferably a plurality of detectable compounds, in particular fluorescently detectable compounds, to be screened for said binding to said plurality of containers;
(iii) incubating the liquid suspension of said F-box protein FBL2, or said fragment, or derivative, or variant thereof, and said compound, preferably said plurality of compounds;
(iv) measuring amounts of detectable compound or fluorescence associated with said F-box protein FBL2, or with said fragment, or derivative, or variant thereof; and
(v) determining the degree of binding by one or more of said compounds to said F-box protein FBL2, or said fragment, or derivative, or variant thereof.

25. A method for producing a medicament comprising the steps of (i) identifying a modulator of neurodegenerative diseases, in particular Alzheimer's disease, by a method according to claim 20 any of claims 20 to 22 and (ii) admixing the modulator with a pharmaceutical carrier.

26. A method for producing a medicament comprising the steps of (i) identifying a compound as an inhibitor of binding between a ligand and the F-box protein FBL2 by a method according to claim 23 and (ii) admixing the compound with a pharmaceutical carrier.

27. A method for producing a medicament comprising the steps of (i) identifying a compound as a binder to the F-box protein FBL2 by a method according to claim 24 and (ii) admixing the compound with a pharmaceutical carrier.

28. A medicament obtainable by the method according to claim 25.

29. A medicament obtained by the method according to claim 25.

30. A protein molecule, said protein molecule being a translation product of a gene coding for the F-box protein FBL2, or a fragment, or derivative, or variant thereof, for use as a diagnostic target for detecting a neurodegenerative disease, preferably Alzheimer's disease.

31. A protein molecule, said protein molecule being a translation product of a gene coding for the F-box protein FBL2, or a fragment, or derivative, or variant thereof, for use as a screening target for reagents or compounds preventing, or treating, or ameliorating a neurodegenerative disease, preferably Alzheimer's disease.

32. An antibody specifically immunoreactive with an immunogen, wherein said immunogen is a translation product of a gene coding for the F-box protein FBL2, or a fragment, or derivative, or variant thereof.

33. Use of an antibody of claim 32 for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell.
